(19) United States
(12) Patent Application Publication
(10) Pub. No.: US 2013/0165752 A1
(43) Pub. Date: Jun. 27, 2013

(54) ENDOSCOPE INTEGRATED WITH A LIGHT SOURCE

(71) Applicant: Yi-Chen Chou, Tainan City (TW)

(72) Inventor: Yi-Chen Chou, Tainan City (TW)

(73) Assignee: HIMAX IMAGING LIMITED, Tainan City (TW)

(21) Appl. No.: 13/335,790

(22) Filed: Dec. 22, 2011

(51) Int. Cl. A61B 1/04

(52) U.S. Cl. 600/109

(57) ABSTRACT

The invention is directed to an endoscope having a wafer-level imaging module (WLM) and a light source bonded on a substrate. The endoscope also includes a holder, to which the substrate with the bonded WLM and the bonded light source is attached.

### BACKGROUND OF THE INVENTION

1. Field of the Invention

The present invention generally relates to an endoscope, and more particularly to an endoscope having a distal section made of a wafer-level module integrated with a semiconductor light source.

2. Description of Related Art

An endoscope is an instrument that is capable of being inserted into an organ to examine the interior of the organ. The endoscope generally includes a flexible tube; a light source disposed at a proximal end of the endoscope and the light is delivered via, an optical fiber; and a lens system disposed at a distal end of the endoscope for collecting image that is transferred via an optical fiber.

Due to the miniature dimension of the endoscope, the manufacturing of the lens system requires great effort and thus making the overall cost high and accessibility low. Moreover, infection may probably occur due to inadequate cleaning of the endoscope. As far as the cost and infection are concerned, a single-use disposable endoscope may alleviate the problems mentioned. above.

For the reason that the conventional endoscope is either high-priced or at the risk of infection, a need has arisen to propose a novel endoscope that eliminates the problems mentioned above.

### SUMMARY OF THE INVENTION

In view of the foregoing, it is an object of the embodiment of the present invention to provide an endoscope having a distal section made of a wafer-level imaging module integrated with a light source, thereby reducing the overall cost of the endoscope. The endoscope of the embodiment may be adaptable to disposable use to eliminate possibility of infection.

According to one embodiment, an endoscope includes a substrate, a wafer-level imaging module (WLM), a light source, a tube and a holder. The WLM and the light source are bonded on a first surface of the substrate, and the tube has a distal end coupled to a second surface of the substrate. The substrate with the bonded WLM and the bonded light source is attached to the holder.

### BRIEF DESCRIPTION OF THE DRAWINGS

FIG. 1A and FIG. 1B show a top view and a lateral cross-sectional view, respectively, of a distal section of an endoscope according to one embodiment of the present invention;

FIG. 1C shows a top view of the substrate of FIG. 1B;

FIG. 2 schematically shows a perspective view of the wafer-level module of FIG. 1B; and

FIG. 3A and FIG. 3B show a top view and a lateral. cross-sectional view, respectively, of the distal section of an endoscope according to another embodiment of the present invention.

### DETAILED DESCRIPTION OF THE INVENTION

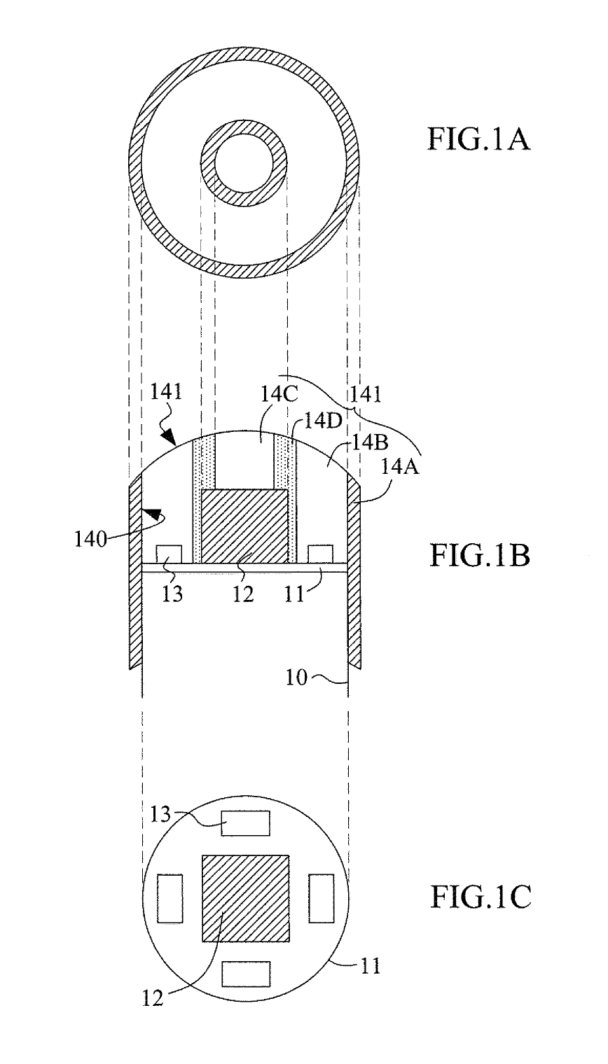

FIG. 1A and FIG. 1B show a top view and a lateral cross-sectional view, respectively, of a distal section of an endoscope according to one embodiment of the present invention. The distal section of the endoscope is disposed at and coupled to a distal end of a tube 10 of the endoscope.

In the embodiment, the distal section primarily includes a substrate 11, a wafer-level imaging module (or wafer-level module, WLM, for short) 12, a light source 13 and a holder 14. Specifically, the WLM 12 and the light source 13 are bonded on a first (top) surface of the substrate 11 (such as a printed circuit board, PCB), and the substrate 11 with the bonded WLM 12 and the light source 13 is then attached to the holder 14.

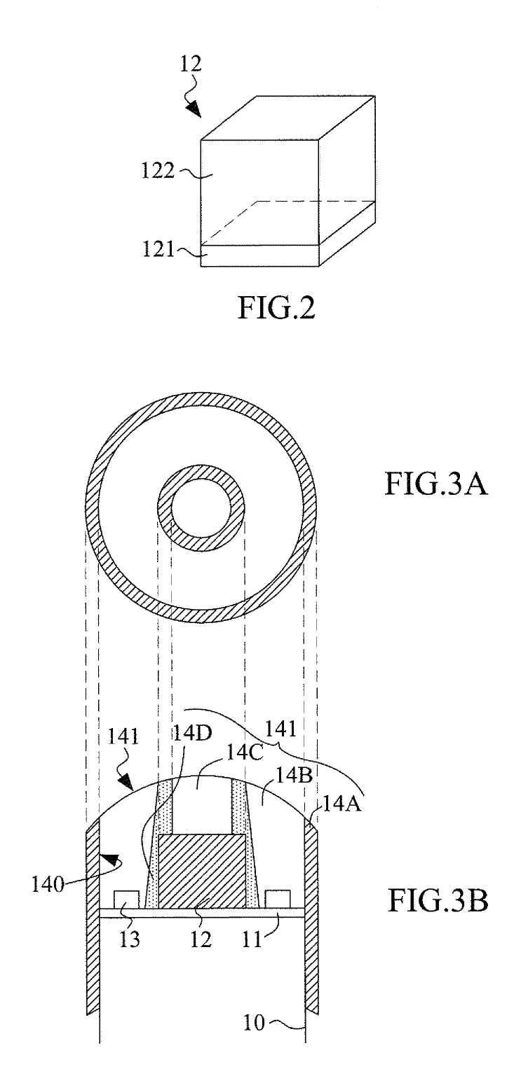

FIG. 2 schematically shows a perspective view of the WLM 12. In the embodiment, the WLM 12 includes a wafer-level image sensor 121. and wafer-level optics (WLO) 122. The wafer-level image sensor 121 is situated facing the substrate 11, and may be, but not limited. to, a complementary metal oxide semiconductor (CMOS) image sensor (commonly abbreviated as CIS). The wafer-level optics 122, such as a lens, is situated away from the substrate 11, and may be made of, but not limited to, glass. The wafer-level image sensor 121 and the wafer-level optics 122 may be bonded together, for example, with an adhesive. Compared to the conventional endoscope, the endoscope of the present embodiment makes use of the mass-productivity and low cost of semiconductor technique to manufacture the imaging system of the endoscope, and is adaptable to the disposable endoscope to eliminate possibility of infection. Wafer-level (or wafer-scale) module is a technique of fabricating miniaturized optics such as lens module or camera module at the wafer level using semiconductor techniques, and details of manufacturing the wafer-level imaging module 12 may be referred, for example, to U.S. Pat. No. 7,564,496 to Wolterink et al., entitled. “Camera device, method. of manufacturing a camera. device, wafer scale package,” the disclosure of which is incorporated herein by reference.

In the embodiment, the light sources 13 may include at least one semiconductor light source such as a light-emitting diode (LED) surrounding the WLM 12 as exemplified in FIG. 1C, a top view of the substrate 11, in which four LEDs are equidistantly disposed on the perimeter of the substrate 11. In one exemplary embodiment, the LEDs 13 and the WLM 12 are bonded on the PCB 11 by reflow soldering. The reflowed PCB 11 is then firmly attached to the inner surface of the holder 14. Finally, the distal end of the tube 10 is coupled to a second (bottom) surface of the PCB 11.

In the embodiment, the holder 14 includes an outer wall 14A and an inner light guide 14B. Specifically, the outer wall 14A constitutes an outer portion, of the holder 14, and is usually made of opaque material. The inner light guide 14B constitutes an inner portion of the holder 14, and is made of transparent material such as plastics (e.g., acrylics). The inner light, guide 14B of the embodiment facilitates light transferred from the light source 13 and then emanated out of the light outlet 141 of the distal section. The holder 14 may have an opening 14C situated above and aligned with the WLM 12. Compared to the conventional endoscope having a light source situated at a proximal end of the endoscope, in which the generated light is delivered, through optical fiber along the entire tube, the endoscope of the present embodiment thus has higher light emission efficiency and lower cost (due to lack of the optical fiber) it is noted that the inner sidewall 140 of the outer wall 14A may be selectively coated with reflective material, thereby further improving the light transferred through the inner light guide 14B. Similarly, the outer sidewall 120 of the WLM 12 may be selectively coated with reflective material, thereby further improving the light transferred through the inner light guide 14B.

The embodiment may further include a shield 14D, disposed between the inner light guide 141B and the WLM 12, used to block scattered light, from entering the wafer-level image sensor 121 of the WLM 12. The shield 14D may be made, for example, of anti-reflective or black material in the embodiment.

FIG. 3A and FIG. 3B show a top view and a lateral cross-sectional view, respectively, of the distal section of an endoscope according to another embodiment of the present invention. The present embodiment is the same as the previous embodiment of FIG. 1B except that the shield 14D has a slant wall facing the inner light guide 14B, therefore forming a lead angle between the slant wall and the outer surface of the WLM 12, assumed that the outer surface of the WLM 12 is perpendicular to the substrate 11. Accordingly, the inner light guide 14B near the light source 13 has a (cross-sectional) area smaller than the (cross-sectional) area of the light, outlet 141. Therefore, light emission uniformity may be substantially improved.

Although specific embodiments have been illustrated and described, it will be appreciated. by those skilled in the art that various modifications may be made without departing from the scope of the present invention, which is intended to be limited solely by the appended claims.

What is claimed is:

1. An endoscope comprising:
a substrate;
a wafer-level imaging module (WLM) bonded on a first surface of the substrate;
a light source bonded on the first surface of the substrate;
a tube having a distal end coupled to a second surface of the substrate; and
a holder, to which the substrate with the bonded WLM and the bonded light source is attached.

2. The endoscope of claim 1, wherein the WLM comprises:
a wafer-level image sensor; and
a wafer-level optics bonded with the wafer-level image sensor;
wherein the wafer-level image sensor is situated facing the substrate, and the wafer-level optics is situated away from the substrate.

3. The endoscope of claim 2, wherein the wafer-level image sensor comprises a complementary metal oxide semiconductor (CMOS) image sensor.

4. The endoscope of claim 2, wherein the wafer-level optics comprises a lens.

5. The endoscope of claim 4, wherein the lens is made of glass.

6. The endoscope of claim 1, wherein the lightsource comprises at least one semiconductor light source.

7. The endoscope of claim 6, wherein the semiconductor light source comprises a light-emitting diode.

8. The endoscope of claim 1, wherein the substrate comprises a printed circuit board.

9. The endoscope of claim 1, wherein the holder comprises:
an outer wall; and
an inner light guide disposed within the outer wall, the inner light guide facilitating light transferred from the light source and then emanated out of a light outlet.

10. The endoscope of claim 9, wherein the outer wall is made of opaque material, and the inner light guide is made of transparent material.

11. The endoscope of claim 9, wherein the holder has an opening situated above the WLM.

12. The endoscope of claim 9, wherein an inner sidewall of the outer wail is coated with reflective material.

13. The endoscope of claim 9, wherein, an outer sidewall of the WLM is coated with reflective material.

14. The endoscope of claim 9, further comprising a shield disposed between the inner light guide and the WLM, used to block scattered from entering the WLM.

15. The endoscope of claim 14, wherein the shield comprises anti-reflective material.

16. The endoscope of claim 14, wherein the shield has a slant wall facing the inner light guide such that the inner light guide near the light source has a cross-sectional area smaller than a cross-sectional area of the light outlet.
